(19) United States
(12) Patent Application Publication
(10) Pub. No.: US 2013/0150256 A1
(43) Pub. Date: Jun. 13, 2013

(54) NOVEL MICRORNAS FOR THE DETECTION AND ISOLATION OF HUMAN EMBRYONIC STEM CELL-DERIVED CARDIAC CELL TYPES

(71) Applicants: Jane Synnergren, Lidkoping (SE); Peter Sartipy, Gothenburg (SE)

(72) Inventors: Jane Synnergren, Lidkoping (SE); Peter Sartipy, Gothenburg (SE)

(21) Appl. No.: 13/703,525

(22) PCT Filed: Jun. 14, 2011
(86) PCT No.: PCT/EP2011/059785

§ 371 Date: Feb. 27, 2013

Related U.S. Application Data
(60) Provisional application No. 61/353,077, filed on Jun. 9, 2010.

(30) Foreign Application Priority Data
Jun. 11, 2010 (DK) PA 2010 00513
Sep. 17, 2010 (DK) PA 2010 00829

(51) Int. Cl. C12N 15/113

(52) U.S. Cl. CPC C12N 15/113
USPC 506/9; 435/375; 435/325; 536/24.5

(57) ABSTRACT

The present invention relates to the use of human-derived microRNAs (miRNAs) as targets for the identification of cardiac and cardiac-like cell types. In particular, it relates to a specific set of miRNAs which have been found to be correlated to cardiac differentiation and can act to up or downregulate a number of putative mRNA targets to guide differentiation and also act as markers for a cardiac phenotype. In addition, it also relates to the use of these miRNAs as tools for the isolation, selection, purification and characterisation of cardiac and cardiac-like cells and tissues. The invention also encompasses the possible use of these miRNAs in the differentiation and maturation of cardiac or cardiac-like cell types.

### TECHNICAL FIELD

The present invention relates to the use of human-derived microRNAs (miRNAs) as targets for the identification of cardiac and cardiac-like cell types. In particular, it relates to a specific set of miRNAs which have been found to be correlated to cardiac differentiation and can act to up or downregulate a number of putative mRNA targets to guide differentiation and also act as markers for a cardiac phenotype. In addition, it also relates to the use of these miRNAs as tools for the isolation, selection, purification and characterisation of cardiac and cardiac-like cells and tissues. The invention also encompasses the possible use of these miRNAs in the differentiation and maturation of cardiac or cardiac-like cell types.

### BACKGROUND OF THE INVENTION

miRNAs

MicroRNAs (miRNAs) are small, non-coding RNA molecules which can regulate gene expression post-transcriptionally, through binding to the 3′UTR of target mRNAs. miRNAs bind to mRNAs and negatively regulate their expression, either by repression of translation or by degradation of the mRNA sequence (gene silencing). Increased expression levels of miRNAs can also result in up-regulation of previously suppressed target genes either directly, by decreasing the expression of inhibitory proteins and/or transcription factors, or indirectly, by inhibiting the expression levels of inhibitory miRNAs. Depending on the state of the cell, miRNAs have been observed to moderate the translation of target mRNA by regulation of their stability. In addition, studies have shown that combinatorial regulation by miRNAs is common. This means that sets of miRNAs together can regulate one or several genes, but also that one miRNA may regulate sets of different genes. Although the target genes and hence the downstream biological functions of most miRNAs have yet to be identified, it is estimated that miRNAs may regulate up to 30% of the genes present in the human genome.

Genes coding for miRNAs are initially transcribed by RNA pol II into long primary miRNAs, which are then further processed into pre-miRNAs of around 80 bps by the RNase III enzyme Drosha. These are further shortened by the RNase II enzyme Dicer which transforms them into duplex miRNAs of around 22 bp. After these miRNA duplexes are unwound to produce mature miRNAs they are incorporated into a ribonucleoprotein complex (miRNP). (Hutvagner, G and Zamore, P. D. 2002). These complexes can lead to downregulation of target gene expression through two distinct mechanisms, either through translational inhibition or through cleavage of target mRNA. Where there is near perfect complementarity between the miRNA and the target gene, cleavage of the target gene will occur with subsequent degradation of the target RNA. In the case of only partial complementarity, translational inhibition will occur instead (Hutvagner, G and Zamore, P. D. 2002).

miRNAs and mRNA Target Pathways

Identifying the targets of miRNAs is still somewhat problematic, largely because most mammalian miRNAs form only imperfect base pairs with their target mRNA sequences. Apart from the initial seed sequence of the miRNA comprising nucleotides from positions 2 to 8, several other variables can also influence pairing and complementarity including RNA secondary structure, site accessibility and interactions with various mRNA binding proteins. Normally a given miRNA will bind to the 3′UTR region of a target mRNA, but miRNAs can have numerous high and low affinity targets and can also indirectly modulate the activity and binding of other miRNAs in turn. This suggests that miRNAs act in a subtle manner to dampen or heighten expression of certain target genes and allow for correct cellular development and function. In contrast they can also have more profound effects as is the case with the cardiac-specific miRNA miR-208, which regulates a group of transcriptional repressors essential for correct development of slow muscle tissue during cardiac development (van Rooij, E et al. 2007).

The actual identification of miRNA targets can be achieved through the use of software packages such as microT (http://diana.cslab.ece.ntua.gr/) which will analyse a miRNA sequence and highlight putative mRNA target sequences. However, validation of these targets and correlating them with specific miRNA phenotypes is still a challenging area. One way to test miRNA target binding is to rely on reporter gene assays, whereby a reporter gene such as GFP or lacZ is linked to the 3′UTR containing the putative miRNA binding site. Repression of reporter gene mRNA activity by miRNA binding can then be measured, although it is important to remember that such an assay may not always reflect the true picture of events in vivo.

Use of miRNA and RNA Analogues in Therapy and Gene-Repression

Several oligonucleotide approaches have been reported for inhibition of miRNAs. WO03/029459 (Tuschl) claims oligonucleotides which encode microRNAs and their complements of between 18-25 nucleotides in length which may comprise nucleotide analogues. LNA is suggested as a possible nucleotide analogue, although no LNA containing oligonucleotides are disclosed. Tuschl claims that miRNA oligonucleotides may be used in therapy. WO07112754A claims the use of miRNA analogues as a pharmaceutical medicament to repress the activity of disease-inducing miRNAs.

US2005/0182005 discloses a 24 mer 2′OMe RNA oligoribonucleotide complementary to the longest form of miR 21 which was found to reduce miR 21 induced repression, whereas an equivalent DNA containing oligonucleotide did not. The term 2′OMe-RNA refers to an RNA analogue where there is a substitution to methyl at the 2′ position (2′OMethyl). US2005/0227934 (Tuschl) refers to antimir molecules with upto 50 percent DNA residues. It also reports that antimirs containing 2′ OMe RNA were used against pancreatic microRNAs but it appears that no actual oligonucleotide structures are disclosed.

An alternative approach to this has been reported by Schwarz D. S et al. (2003), in which 2′-O-methyl antisense oligonucleotides, complementary to the mature miRNA could be used as potent and irreversible inhibitors of short interfering RNA (siRNA) and miRNA function in vitro and in vivo in Drosophila and C. elegans, thereby inducing a loss-of-function phenotype. A drawback of this method is the need of high 2′-O-methyl oligonucleotide concentrations (100 micromolar) in transfection and injection experiments, which may be toxic to the animal. This method was recently applied to mice studies, by conjugating 2′-O-methyl antisense oligonucleotides complementary to four different miRNAs with cholesterol for silencing miRNAs in vivo (Krützfedt, J. et al. 2005). These so-called antagomirs were administered to mice by intravenous injections. Although these experiments resulted in effective silencing of endogenous miRNAs in vivo, which was found to be specific, efficient and long-lasting, a major drawback was the need of high dosage (80 mg/kg) of 2′-0-Me antagomir for efficient silencing.

miRNAs During Cardiac Development and Disease

The role of miRNA during cardiac development has been examined by a number of research group including Srivastava, D. et al, (1997), Zhao, Y. et al. (2005) and has further been published in US2010010073A and WO09106367A.

Recently, the involvement of miRNAs in cardiac remodelling has been examined by e.g. van Rooij, E. et al (2006) and published in WO 2009/058818 A2).

### SUMMARY OF THE INVENTION

The invention involves the use of a set of human-derived microRNAs (miRNAs) which are up or downregulated during mammalian cardiogenesis- The miRNAs disclosed herein may act as markers for the identification of cardiac or cardiac-like cell types. In addition, the invention also incorporates the use of these microRNAs as tools for the isolation, selection and characterisation of cardiac, or cardiac-like cells and tissues. The invention further encompasses the possible use of these miRNAs in promoting the differentiation and maturation of cardiac, cardiac-like or cardiac progenitor cell types and in the identification of cardiac disease states.

Definitions

As used herein, the term “microRNA” (“miRNA”) refers to any type of interfering RNAs, including but not limited to, endogenous microRNAs and artificial microRNAs (e.g. synthetic miRNAs). Endogenous microRNAs are small RNAs encoded within the genome which are capable of regulating the post-transcriptional expression of one or more target genes through translational repression and/or destabilisation of protein-coding mRNAs. An artificial microRNA can be any type of RNA sequence, other than endogenous microRNA, which is capable of modulating the activity of an mRNA molecule. Examples of microRNAs include any RNA that is a fragment of a larger RNA or is a miRNA, siRNA, stRNA, sncRNA, tncRNA, snoRNA, smRNA, snRNA or other small non-coding RNA. (see US Patent Applications 20050272923, 20050266552, 20050142581, 20050075492). The term microRNA may also encompass non-naturally occurring nucleotides which have modified sugar moieties, such as bicyclic nucleotides or 2′ modified nucleotides, such as 2′ substituted nucleotides.

As used herein, the term “nucleotide” refers to a glycoside comprising a sugar moiety, a base moiety and a covalently linked phosphate group and covers both naturally occurring nucleotides, such as DNA or RNA, preferably DNA, and non-naturally occurring nucleotides comprising modified sugar and/or base moieties, which are also referred to as “nucleotide analogues” herein.

“Nucleotide analogues” are variants of natural nucleotides, such as DNA or RNA nucleotides, by virtue of modifications in the sugar and/or base moieties. Analogues could in principle be merely “silent” or “equivalent” to the natural nucleotides in the context of the oligomer, i.e. have no functional effect on the way the oligomer works to inhibit target gene expression. Such “equivalent” analogues may nevertheless be useful if, for example, they are easier or cheaper to manufacture, or are more stable to storage or manufacturing conditions, or represent a tag or label. Preferably, however, the analogues will have a functional effect on the way in which the oligomer works to inhibit expression; for example by producing increased binding affinity to the target and/or increased resistance to intracellular nucleases and/or increased ease of transport into the cell. Specific examples of nucleoside analogues are described by e.g. Freier, S. M. & Altmann, K. H., (1997), and Uhlmann, E. (2001).

As used herein the term “isolated cell”, refers to a cell that is in an environment different from that in which the cell naturally occurs, e.g. where the cell naturally occurs in a multicellular organism, and the cell is removed from the multicellular organism, the cell is “isolated”. The term “isolated” can also be taken to mean that the cell in question is enriched in abundance within a population of cells without actually being physically separated from the surrounding cells. An isolated genetically modified host cell can be present in a mixed population of genetically modified host cells, or in a mixed population comprising genetically modified cells and host cells that are not genetically modified. Similarly, a hPS-derived cell could be isolated from a heterogeneous mix of hPS and differentiated cell types where the differentiated cell types no longer retain the characterisitic pluripotency.

As used herein, “cardiac”, “cardiac-like” and “cardiac progenitor” refer to cells or tissues which express at least one marker from the following list (and preferably three to five markers): Cardiac troponin I (cTnI), cardiac troponin T (cTnT), sarcomeric myosin heavy chain (MHC), GATA-4, Nkx2.5, N-cadherin, β1-adrenoceptor (β1-AR), ANF, the MEF-2 family of transcription factors, creatine kinase MB (CK-MB), myoglobin, or atrial natriuretic factor (ANF)

Such cells or tissues should also show absence of expression of at least one (and preferably two to four) of the following markers of undifferentiated tissue: Oct3/4, TRA1-60, SSEA4, Sox2.

Throughout this disclosure, techniques that refer to “cardiac”, “cardiac-like” and “cardiac progenitor” can be taken to apply equally to cells at any stage of cardiomyocyte ontogeny without restriction, as defined above, unless otherwise specified. The cells may or may not have the ability to proliferate or exhibit contractile activity.

As used herein, the term “reference”, “reference sample” or “reference value” refer to a sample or value in which a specific threshold value is given. Hence a measured value may be higher or lower than the reference value and thereby provide guidance for interpreting the measured value. A reference may thus be the gene or miRNA expression level in a relevant type of tissue, such as undifferentiated pluripotent stem cells or developed cardiac tissue.

As used herein, “human pluripotent stem cells” (hPS) refers to cells that may be derived from any source and that are capable, under appropriate conditions, of producing human progeny of different cell types that are derivatives of all of the 3 germinal layers (endoderm, mesoderm, and ectoderm). hPS cells may have the ability to form a teratoma in 8-12 week old SCID mice and/or the ability to form identifiable cells of all three germ layers in tissue culture. Included in the definition of human pluripotent stem cells are embryonic cells of various types including human embryonic stem (hES) cells, (see, e.g., Thomson, J. A. et al. (1998), as well as induced pluripotent stem cells (see, e.g. Yu, J. et al., (2007); Takahashi, K. et al., (2007). The various methods and other embodiments described herein may require or utilise hPS cells from a variety of sources. For example, hPS cells suitable for use may be obtained from developing embryos. Additionally or alternatively, suitable hPS cells may be obtained from established cell lines and/or iPS (induced pluripotent stem cells).

Further, cells applicable to the methods disclosed in present invention may encompass any cell differentiated to any stage between undifferentiated to fully differentiated (mature) cardiac cell.

### DETAILED DESCRIPTION OF INVENTION

The present invention involves a defined set of human-derived microRNAs (miRNAs) which have been identified and whose expression levels have been found to be altered (either up or downregulated) during the differentiation of human pluripotent stem cells towards cardiac-like cell types.

One aspect of the invention relates to a method for isolating stem cells exhibiting cardiac or cardiac-like characteristics from a heterogeneous mixed-cell population using one or more microRNAs, the method comprising the steps:
a) analysing the level of one or more intracellular miRNAs
b) optionally, removing unwanted cells
c) mechanically and/or enzymatically isolating the cells showing signs of cardiac differentiation
d) optionally, adding substances such as e.g. growth factors or differentiating agents to the growth medium to promote differentiation towards a cardiomyocyte cell fate.

The one or more miRNA may be linked to a defined differentiation step and may be selected from one of the sets A to H as outlined in table 1 herein.

Thus, an aspect of the invention relates to a method wherein at least one, such as e.g. at least 2 or at least 3 or more of the miRNAs belongs to the group consisting of the miRNAs which are upregulated and/or downregulated in CMC3w (Tables 1.i and/or 1.ii) or CMC7w (Tables 1.iv and/or 1.iii) or foetal heart (Tables 1.vi and/or 1.v) or adult heart (Tables 1.viii and/or 1.vii) or CMC3w and CMC7w (Tables 1.ix) and/or 1.x) or CMC3w, CMC7w and Foetal Heart (FH) (Tables 1.xi) and/or 1.xii) or CMC3w, CMC7w, Foetal Heart (FH) and Adult Heart (AH) (Tables 1.xiii) and/or 1.xiv).)

More specifically the one or more miRNA may be selected from the group consisting of miR-378, miR-152, miR-1297, miR-208a, miR-208b, miR-451, miRPlus-E1117, miRPlus-E1141, miRPlus-E1202, miR-25*, miRPlus-E1038. In an aspect of the invention, the level of the one or more miRNAs is compared to a reference level.

In a further aspect, the miRNA level may be measured by an expression construct and wherein the miRNA encoding sequence or promoter sequence is fused to an exogenous reporter gene sequence. Alternative ways of measuring the miRNA level comprise methods such as qPCR, rtPCR, hybridisation techniques, DNA or RNA ligand techniques etc. Accordingly, an aspect of present invention relates to a genetically modified cell, containing a nucleotide construct where the miRNA encoding sequence or promoter sequence is fused to a reporter gene sequence. The reporter gene sequence may be of non-human origin. The nucleotide construct may be stably integrated into the cell genome or present as an individual, non genomically integrated construct in the cell. As discussed herein, the miRNA expression construct may be transiently expressed

In a further aspect, present invention relates to a cell obtained by the method as described in any of the methods disclosed herein, and in particular to a cell such as e.g. a stem cell, exhibiting cardiac or cardiac-like characteristics growing in an in vitro environment comprising on or more miRNAs selected from any of the groups A-N or tables 1.i) to 1.xiv) miRNA antagomirs targeting on or more miRNAs selected from any of the groups A-N or tables 1.i) to 1.xiv).

In a further aspect the one or more miRNAs comprised in the in vitro environment is exogenously added.

Based on expression studies on material from adult heart (AH), foetal heart (FH), and 3 week old and 7 week old cardiomyocyte tissue derived from human pluripotent stem cells (CMC3w, CMC7w), used in comparison with non-cardiac undifferentiated human pluripotent stem cells (UD), the inventors of present invention have found 8 sets of miRNAs that show profound linkage to cardiac development during specific developmental stages as outlined below.

TABLE 1A
Sets of miRNA

| Set of | Up- | Down- | | List of | Cardiac |
|---|---|---|---|---|---|
| miRNA | regulated | regulated | Count | miRNA | Development |
| A | | CMC-w3- | 70 | Table 1 | Early |
| | | DOWN | | i) | stage |
| B | CMCw3- | | 147 | Table 1 | |
| | UP | | | ii) | |
| C | | CMCw7- | 70 | Table 1 | Early to |
| | | DOWN | | iii) | medium stage |
| D | CMCw7- | | 171 | Table 1 | |
| | UP | | | iv) | |
| E | | FH-DOWN | 149 | Table 1 | Early to |
| | | | | v) | late stage |
| F | FH-UP | | 173 | Table 1 | |
| | | | | vi) | |
| G | | AH-DOWN | 38 | Table 1 | Early to |
| | | | | vii) | adult stage |
| H | AH-UP | | 84 | Table 1 | |
| | | | | viii) | |
| I | | CMC-w3- | 48 | Table 1 | Early to |
| | | DOWN + | | x) | medium stage |
| | | CMC-w7- | | | |
| | | DOWN | | | |
| J | CMCw3- | | 130 | Table 1 | |
| | UP + | | | ix) | |
| | CMCw7- | | | | |
| | UP | | | | |
| K | | CMC-w3- | | Table | Early to |
| | | DOWN + | | 1.xii) | late stage |
| | | CMC-w7- | | | |
| | | DOWN + | | | |
| | | FH-DOWN | | | |
| L | CMCw3- | | | Table | |
| | UP + | | | 1.xi) | |
| | CMCw7- | | | | |
| | UP + | | | | |
| | FH-UP | | | | |
| M | | CMC-w3- | 24 | Table | Early to |
| | | DOWN + | | 1.xiv) | adult stage |
| | | CMC-w7- | | | |
| | | DOWN + | | | |
| | | FH-DOWN + | | | |
| | | AH-DOWN | | | |
| N | CMCw3- | | 61 | Table | |
| | UP + | | | 1.xiii) | |
| | CMCw7- | | | | |
| | UP + | | | | |
| | FH-UP + | | | | |
| | AH-UP | | | | |

Using the SAM statistical algorithm, differentially expressed miRNAs in the four samples (CMC3w, CMC7w, FH, and AH) were identified when compared to UD. Table 1 shows the number of up- and down-regulated miRNAs and notably there are more than twice as many up-regulated than down-regulated miRNAs in the CMC samples, indicating the importance of increased miRNA expression of selected miRNAs during cardiac development.

As listed in table 1 above and shown in FIG. 2A, there is a highly concordant miRNA expression pattern in the different samples. The significance threshold applied in all analyses was FDR<0.05. In total, 147 miRNAs were up-regulated in CMC3w and 130 of these (88%) were also up-regulated in CMC7w (FIG. 2A). In total, 70 miRNAs were down-regulated in both the CMC3w and the CMC7w respectively, and 48 of these (69%) are overlapping between these two samples. In the human heart tissue samples, fewer miRNAs were detected as differentially expressed compared to the situation in the CMCs. Significantly more miRNAs were differentially expressed in FH than in AH, suggesting that a less mature tissue may have a more active miRNA transcriptional program. Notably, the overlap between FH and AH was close to 100% of AH, 79 out of 84 up-regulated miRNAs in AH are also up-regulated in FH, and 37 out of 38 down-regulated miRNAs in AH are also down-regulated in FH (FIG. 2A). Interestingly, the overlap between the CMCs and the heart tissue samples was large and 61 of the 79 miRNAs (77%) that were up-regulated in the heart tissues were also up-regulated in both CMC groups. Regarding down-regulated miRNAs, 24 of the 37 miRNAs (65%) that were down-regulated in both FH and AH were also down-regulated in the CMC groups (FIG. 2A). Only the intersection of miRNAs that were significantly up- or down-regulated in all four cell- and tissue samples (CMC3w, CMC7w, FH, and AH) were finally selected for further studies regarding their potential effect on target mRNA expression, and these are detailed in Tables 1i)-1.xiv)

Thus, an aspect of the invention relates to method for detecting and characterising differentiating stem cells as cardiac or cardiac-like cells within a stem cell population, the method comprising the steps
a) analysing the cellular level of one or more miRNAs.

Accordingly, miRNA is used as a marker of cardiomyocyte differentiation.

In a similar way as for the miRNAs, the intersections of significantly up- or down-regulated mRNAs (FDR<0.05) in the four cell- and tissue samples were determined and the results 35 are shown in FIG. 2B and table 1B below.

TABLE 1B
Set of mRNA regulated during cardiogenesis.

| Set of | Up- | Down- | | Cardiac |
|---|---|---|---|---|
| mRNA | regulated | regulated | Count | Development |
| a′ | | CMC-w3- | 3520 | Early |
| | | DOWN | | stage |
| b′ | CMCw3- | | 3138 | |
| | UP | | | |
| c′ | | CMCw7- | 5513 | Early to medium |
| | | DOWN | | stage |
| d′ | CMCw7- | | 4827 | |
| | UP | | | |
| e′ | | FH-DOWN | 5518 | Early to |
| f′ | FH-UP | | 4194 | late stage |
| g′ | | AH-DOWN | 6323 | Early to |
| h′ | AH-UP | | 4871 | adult stage |
| i′ | | CMC-w3- | 3113 | Early to medium |
| | | DOWN + | | stage |
| | | CMC-w7- | | |
| | | DOWN | | |
| j′ | CMCw3- | | 1871 | |
| | UP + | | | |
| | CMCw7- | | | |
| | UP | | | |
| k′ | | CMC-w3- | | Early to |
| | | DOWN + | | late stage |
| | | CMC-w7- | | |
| | | DOWN + | | |
| | | FH-DOWN | | |
| l′ | CMCw3- | | | |
| | UP + | | | |
| | CMCw7- | | | |
| | UP + | | | |
| | FH-UP | | | |
| m′ | | CMC-w3- | 2613 | Early to |
| | | DOWN + | | adult stage |
| | | CMC-w7- | | |
| | | DOWN + | | |
| | | FH-DOWN + | | |
| | | AH-DOWN | | |
| n′ | CMCw3- | | 1177 | |
| | UP + | | | |
| | CMCw7- | | | |
| | UP + | | | |
| | FH-UP + | | | |
| | AH-UP | | | |

In total, 3,138 mRNAs were up-regulated in CMC3w and 1,871 (60%) of these were also up-regulated in CMC7w. Notably, there was a pronounced increase (54%) in the number of up-regulated mRNAs in CMC7w compared to CMC3w. In contrast to what was observed in the miRNA data, there were more up-regulated mRNAs in the heart tissue samples than in the hESC-derived CMC samples. All together, 4,194 mRNAs were up-regulated in the AH and 2,165 (52%) of these were also up-regulated in FH. A comparison of mRNA expression between heart tissue and CMCs demonstrate an overlap of 63% between these two groups, meaning that 1,177 of the 1,871 mRNAs that were up-regulated in the CMCs were also up-regulated in both FH and AH. In contrast to what was observed in the miRNA data, there were more down-regulated than up-regulated mRNAs in the cell- and tissue samples. Generally, higher overlaps between pairs of samples (CMC3w-CMC7w and FH-AH) were observed for the down-regulated mRNAs (˜80%) than for the up-regulated mRNAs (˜60%). Interestingly, there were more than twice as many down-regulated (2,613) than up-regulated (1,177) mRNAs that overlapped between all four samples, which is an opposite pattern of what is observed in the miRNA data.

Interestingly, the results demonstrate that on average ˜29% of the up-regulated miRNA target genes and ˜40% of the down-regulated miRNA target genes (Tables 1.i) to 1.xiv)) were differentially expressed in all four cell- and tissue samples. Of particular note is that among the target genes displaying differential expression, both up- and down regulated genes were observed. As shown in Table 1B, there were large variations both in the number of putative target genes identified and in the target gene expression patterns across different miRNAs. No target genes were identified for six of the up-regulated miRNAs (miR-208a, miR-208b, miR-451, miRPlus-E1117, miRPlus-E1141, miRPlus-E1202) and two of the down-regulated miRNAs (miR-25*, miRPlus-E1038) using 12 as the score threshold.

In one aspect of the analysis, to increase the validity of the results, this analysis was restricted to only include target genes of miRNAs that were significantly up- or down-regulated in all four samples and only mRNAs that were significantly up- or down-regulated in all four cell- and tissue samples qualified as differentially expressed. Using the DAVID Bioinformatic resource (http://david.abcc.ncifcf.gov/) and in total 608 target genes, which were defined as differentially expressed in previous steps as input, significantly overrepresented annotations were determined.

Interestingly, several of these relate to cardiac function and cardiac development. FIG. 3 shows pie charts of the overrepresented annotations in the three categories BP, MF and CC. Among the enriched annotation terms for the BP category were ‘heart development’, ‘heart contraction’, ‘organ morphogenesis’, and ‘regulation of transcription’ (FIG. 3A). For the MF category, ‘ion binding activities’ and ‘protein kinas activity’ were the two major groups of enriched annotations (FIG. 3B) and for the category CC ‘various parts of the nucleus’ and Intracellular organelles' were identified as significantly enriched (FIG. 3C).

Hence, the inventors have found that the miRNAs as disclosed herein are applicable as means for modulating the differentiation of pluripotent stem cells towards a cardiac lineage.

Thus, one or more miRNAs whose expression is significantly upregulated during cardiogenesis can be selected and its expression level artificially increased in any undifferentiated pluripotent cell types in order to drive the differentiation of the cell towards a cardiac phenotype. This subject method involves:

a) introducing into a stem cell a microRNA nucleic acid, or a microRNA-encoding nucleic acid from the previously described set of microRNAs (see any of the Tables 1.i) to 1.xiv)), thereby increasing the number of cardiac or cardiac progenitor cells; and

b) introducing into the cardiac progenitor cells a microRNA nucleic acid or a microRNA-encoding nucleic acid, thereby inducing differentiation of the cardiac progenitor cells into cardiomyocytes. In a preferred embodiment, the miRNA chosen to modulate cardiac development and maturation will be chosen from the following list: miR-152, miR-378, miR-1297, miRPLUS-E1202, miRPLUS-C1049

Thus, an aspect of the invention relates to a method for promoting the development and/or maturation of stem cells towards cardiac progenitor or cardiomyocyte-like cells in vitro, by manipulating the intracellular level of one or more miRNAs.

Depending in the developmental stage, miRNAs that are characteristic for a specific developmental stage or are up- or downregulating through one or more developmental stages may be applied.

Thus a further aspect of the invention relates to a method wherein at least one such as e.g. at least 2 or at least 3 or more of the miRNAs belongs to the group consisting of the miRNAs which are upregulated or downregulated in CMC3w (Tables 1.i and 1.ii) or CMC7w (Tables 1.iv and/or 1.iii) or foetal heart (Tables 1.vi and/or 1.v) or adult heart (Tables 1.viii and/or 1.vii) or CMC3w and CMC7w (Tables 1.ix and 1.x) or CMC3w, CMC7w and Foetal Heart (FH) (Tables 1.xi) and 1.xii) or CMC3w, CMC7w, Foetal Heart (FH) and Adult Heart (AH) (Tables 1.xiii and 1.xiv). In a further aspect of the invention, the one or more miRNA may be selected from the group consisting of miR-378, miR-152, miR-1297, miR-208a, miR-208b, miR-451, miRPlus-E1117, miRPlus-E1141, miRPlus-E1202, miR-25*, miRPlus-E1038 or miRPlus -1047.

Hence an aspect of the invention relates to the miRNA sequences:

| | |
|---|---|
| miRPlus-E1117: | AAGACGAGAAGACCCUAUGGAGCUU |
| miRPlus-E1141: | GCGUAAAGAGUGUUUUAGAUCACCC |
| miRPlus-E1202: | GAUUAGGGUGCUUAGCUGUUAACU |
| miRPlus-E1038: | GCAUGAGUGGUUCAGUGGU |
| miRPlus-E1047: | GCUGAGUGAAGCAUUGGACUGU |

The sequences may be used for detecting, characterising, isolating or differentiating hPS cells, as well as in diagnostics, drug discovery, toxicity testing or drug development.

As disclosed herein, the development and/or maturation of stem cell towards cardiac or cardiomyocyte-like cells is done through the up or downregulation of one or more endogenous miRNAs, through the introduction into the cell of one or more miRNAs, through the introduction into a cell of one or more miRNA nucleotide analogues, through introducing into a stem cell or progenitor cell a miRNA antagomir. Further, the miRNAs can be introduced as a nucleotide construct such as an expression construct, as a solution of miRNAs or as viral constructs. The miRNA material may be a purified miRNA sequence.

In any of the aspects mentioned herein, the miRNA or miRNA antagomir may be a nucleotide analogue or a purified miRNA sequence. In some aspects of the invention, the nucleic acid encoding the miRNA is linked to an inducible promoter sequence.

As noted above, in some embodiments, an aspect of the invention refers to a method which involves introducing into a stem cell or a progenitor cell (or a population of stem cells or progenitor cells) a miRNA-encoding nucleic acid. In some embodiments, a subject method involves introducing into a stem cell or a progenitor cell (or a population of stem cells or progenitor cells) one or more nucleic acids comprising nucleotide sequences encoding an miRNA. Suitable nucleic acids comprising miRNA-encoding nucleotide sequences include expression vectors (“expression constructs”), where an expression vector comprising a miRNA-encoding nucleotide sequence is a “recombinant expression vector.” In some embodiments, the expression construct is a viral construct, e.g., a recombinant adeno-associated virus construct (see, e.g., U.S. Pat. No. 7,078,387), a recombinant adenoviral construct, a recombinant lentiviral construct, etc.

Suitable expression vectors include, but are not limited to, viral vectors (e.g. viral vectors based on vaccinia virus; poliovirus; adenovirus (see, e.g.,; WO 94/12649, WO 93/03769; WO 93/19191; WO 94/28938; WO 95/11984 and WO 95/00655); adeno-associated virus (see, e.g., Flannery, J. G. et al. (1997); WO 93/09239), SV40; herpes simplex virus; human immunodeficiency virus (see, e.g., Miyoshi, H. et al. (1997); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

Numerous suitable expression vectors are known to those of skill in the art, and many are commercially available. The following vectors are provided by way of example; for eukaryotic host cells: pXT1, pSG5 (Stratagene), pSVK3, pBPV, pMSG, and pSVLSV40 (Pharmacia). However, any other vector may be used so long as it is compatible with the host cell.

Depending on the host/vector system utilized, any of a number of suitable transcription and translation control elements, including constitutive and inducible promoters, transcription enhancer elements, transcription terminators, etc. may be used in the expression vector (see e.g., Bitter, G. A. et al. (1987)

A miRNA-encoding nucleotide sequence may be operably linked to a control element, e.g., a transcriptional control element, such as a promoter. Likewise, in some embodiments, a miRNA-encoding nucleotide sequence is operably linked to a control element, e.g., a transcriptional control element, such as a promoter. Selection of the appropriate vector and promoter is well within the level of ordinary skill in the art. The expression vector may also contain a ribosome binding site for translation initiation and a transcription terminator. The expression vector may also include appropriate sequences for amplifying expression. In some embodiments, the miRNA-encoding nucleotide sequence is operably linked to an inducible promoter. In some embodiments, the miRNA-encoding nucleotide sequence is operably linked to a constitutive promoter.

Methods of introducing a nucleic acid into a host cell are known in the art, and any known method can be used to introduce a nucleic acid (e.g., an expression construct) into a stem cell or progenitor cell. Suitable methods include, e.g., infection, lipofection, electroporation, calcium phosphate precipitation, DEAE-dextran mediated transfection, liposome-mediated transfection, and the like. Introducing a nucleic acid may also include contacting a host cell with a compound, small molecule, activating RNA, or other agent in order to force expression of the endogenous nucleic acid.

Thus an aspect of the invention relates to a genetically modified cell, containing an nucleotide construct comprising a miRNA selected from any of the groups A-N, where the miRNA encoding sequence or promoter or miRNA target sequence is fused to a reporter gene sequence. The reporter gene sequence may be of non-human origin and the nucleotide construct may be present in the cell as a non-integrated nucleotide sequence such as a vector or is stably integrated into the cell genome of the cell. Further, the miRNA nucleotide construct may be constitutively or transiently expressed.

As discussed above, the miRNA sequences disclosed herein may be added to a cell growing in vitro. Hence present invention relates to a cell exhibiting cardiac or cardiac-like characteristics growing in an in vitro environment comprising miRNAs or miRNA antagomir. The miRNA may be exogenously added as miRNA analogues, double stranded sequences and in all forms described herein. In a particular aspect of the invention, the miRNA is selected from any of the groups A-N or miRNA antagomirs targeting miRNA selected from any of the groups A-N as described above. Further, the cell according may be a pluripotent stem cell, such as e.g., an induced pluripotent stem cell or a blastocyst derived stem cell.

In a further aspect of the invention the identified sets of microRNAs (any of the Tables 1.i) to 1.xiv)) can be used for the diagnosis of heart diseases. In one particularly preferred embodiment, the heart disease is myocardial infarction, heart failure, in particular chronic heart failure and/or cardiac hypertrophy. A further subject matter of the invention is a method for the diagnosis of a heart disease, wherein the method comprises the steps: (a) providing a sample from an individual suspected of suffering from a heart disease; (b) measuring the expression level of at least one sequence selected from any of the Tables 1.i) to 1.xiv) in the sample; wherein an increased or reduced expression level of at least one sequence selected from any of the Tables 1.i) to 1.xiv) compared to a control sample indicates a heart disease or a prevalence or predisposition to a heart disease.

| | | P- | |
|---|---|---|---|
| KEGG pathway^{a} | Count^{b} | value^{c} | Differentially expressed miRNA target genes^{d} |
| hsa04720: | 11 | 0.0002 | PPP1CC. RPS6KA6. PLCB1. PLCB4. PRKX. |
| Long-term | | | CACNA1C. RPS6KA2. CAMK2D. CAMK4. |
| potentiation | | | KRAS. RPS6KA3 |
| hsa04916: | 11 | 0.005 | DVL3. PLCB1. PLCB4. PRKX. MITF. CAMK2D. |
| Melanogenesis | | | ADCY6. GNAI2. FZD5. KRAS. ADCY9 |
| hsa04360: | 13 | 0.006 | LIMK1. EPHB1. ITGB1. KRAS. CFL2. PAK1. |
| Axon guidance | | | UNC5D. NRP1. PLXNA2. NFAT5. SEMA6A. |
| | | | GNAI2. EPHA3 |
| hsa04310: | 14 | 0.007 | DVL3. CCND2. PLCB4. VANGL2. FRAT2. |
| Wnt signaling | | | TP53. PLCB1. PRKX. NFAT5. DAAM1. |
| pathway | | | CAMK2D. SFRP1. FZD5. VANGL1 |
| hsa04912: | 10 | 0.012 | PLCB1. PLCB4. PRKX. CACNA1C. MAP3K4. |
| GnRH signaling | | | CAMK2D. ADCY6. HBEGF. KRAS. ADCY9 |
| pathway | | | |
| hsa04510: | 16 | 0.012 | COL3A1. CCND2. PDPK1. SHC4. IGF1. |
| Focal adhesion | | | ITGA6. ITGB1. KRAS. HGF. ITGB3. PAK1. |
| | | | PPP1CC. RELN. ITGAV. COL1A2. VEGFC |
| hsa04020: | 14 | 0.020 | ATP2A2. RYR2. PLCB4. EDNRA. ADCY9. |
| Calcium signaling | | | ERBB4. SLC25A4. PLCB1. PRKX. CACNA1C. |
| pathway | | | ATP2B4. GNAL. CAMK2D. CAMK4 |
| hsa05216: | 5 | 0.021 | TPM3. CCDC6. TP53. RET. KRAS |
| Thyroid cancer | | | |
| hsa04512: | 9 | 0.023 | COL3A1. RELN. ITGAV. SV2A. COL1A2. |
| ECM-receptor | | | ITGA6. ITGB1. CD44. ITGB3 |
| interaction | | | |
| | | P- | |
| BioCarta pathway | Count | value | Differentially expressed miRNA target genes |
| h_btg2Pathway: | 4 | 0.012 | CNOT7. BTG2. TP53. BTG1 |
| BTG family proteins | | | |
| and cell cycle | | | |
| regulation | | | |
| h_pgc1aPathway: | 4 | 0.026 | PPARGC1A. MEF2A. CAMK4. PPARA |
| Regulation of PGC- | | | |
| 1a | | | |
| h_nfatPathway: | 6 | 0.042 | IGF1. CAMK4. HBEGF. KRAS. HAND1. |
| NFAT and | | | HAND2 |
| Hypertrophy of the | | | |
| heart (Transcription | | | |
| in the broken heart) | | | |

Hence an aspect of the invention relates to a method for diagnosing cardiac abnormalities and disease states in a sample, comprising the detection of alterations in the levels of at least one miRNA within the diseased cells compared to a control group. The samples may be of non-human origin and isolated from the body prior to measurement.

### EXAMPLES

### Example 1

### Generation of Cardiac-Specific microRNAs

Cardiac differentiation was performed as described previously using a previously established human embryonic stem cell line (SA002, Cellartis AB, www.cellartis.com). Briefly, to initiate differentiation 3D cell aggregates were formed in 96-well plates. After 3-5 days, the aggregates were plated onto gelatine-coated culture dishes for further differentiation and maintenance. Spontaneously beating CMCs were harvested at 3- and 7 weeks post-plating by mechanical dissection.

### Example 2

### Isolation of Specific microRNAs from hPS, Adult Cardiac and Foetal Cardiac Cell Types

Total RNA was extracted using Ambion miRVana miRNA isolation kit, preserving small molecules according to the manufacturer's instructions (Ambion, Inc., www.ambion.com). Quantification of nucleic acids was performed on NanoDrop ND-1000 (NanoDrop, www.nanodrop.com). Microarray experiments were conducted in parallel to measure both miRNA and mRNA expression from paired samples. The material consisted of samples of undifferentiated hESCs and hESC-derived CMCs cultured for 3- (CMC3w) and 7 weeks (CMC7w) after onset of differentiation. In addition, samples from fetal heart (FH) and adult heart (AH) (Yorkshire Bioscience, www.york-bio.com) were included as reference material [FIG. 1i)]. The experiments were repeated three times to generate biological replicates, and the human reference RNA material was obtained from three separate batches. The quality of the RNA and cDNA, labeled by in vitro transcription, was verified using an Agilent Bioanalyzer.

To measure the mRNA expression, fragmented cDNA was hybridized at 45° C. for 16 hours to whole transcript Gene ST 1.0 arrays (Affymetrix, www.affymetrix.com). The microarrays were scanned on a GeneChip Scanner 3000 7G (Affymetrix) and expression signals were extracted and normalized by means of the Expression Console™ (Affymetrix) applying the Robust Multichip Average (RMA) normalization method. For the miRNA experiment, samples were labeled using the miRCURY™ LNA Array power labeling kit (Exiqon, www.exiqon.com) and subsequently hybridized at 54° C. for 16 hours to the miRCURY™ LNA array version 11.0 (Exiqon) following the manufacturer's instructions. After hybridization, the microarray slides were scanned using the Agilent G2565BA Microarray Scanner System (Agilent Technologies, www.agilent.com) and the image analysis was carried out using the ImaGene 8.0 software (BioDiscovery, www.biodiscovery.com). The quantified signals were background corrected and normalized using the global Lowess regression algorithm.

### Example 3

### Identification of Differentially Expressed miRNAs and mRNAs

MicroRNAs that were significantly up- or down-regulated in CMC3w, CMC7w, FH, and AH compared to undifferentiated hESCs (UD) were identified using the Significant Analysis of Microarray data (SAM) algorithm ¹⁶. SAM controls for false discovery rate (FDR), and miRNAs with an FDR<0.05 were here defined as differentially expressed, and thus selected for further analysis. The same procedure was applied for identification of differentially expressed mRNA sequences (see Tables 1.i) to 1.xiv)).

### Example 4

### Investigation of the Transcriptional Effects at mRNA level of Up- and Down-Regulated miRNAs

The correlation between miRNA expression and mRNA expression in the CMCs and tissue samples was investigated applying the following approach [FIG. 1ii)]:. (i) Only the miRNAs that were significantly up- or down-regulated in all four samples (CMC3w, CMC7w, FH, and AH) were selected for further investigation. (ii) For each of these miRNAs, putative target genes were identified using the software tool microT v3.0 (http://diana.cslab.ece.ntua.gr/microT/). The parameter target score threshold was set to 12, which is considered as a moderate threshold for identification of target genes for miRNAs. (iii) To explore if the mRNA expression levels of predicted target genes were affected by significant changes in miRNA expression, the number of target genes that were identified as significantly up- or down-regulated in the mRNA data, was calculated. For an mRNA sequence to qualify as differentially expressed it had to be differentially expressed in all four cells- and tissue samples. Using these restrictions, the percentages of up- and down-regulated target genes of each miRNA were calculated.

### Example 5

### Over-Representation of Gene Pathways Among the Differentially Expressed Target Genes

KEGG and BioCarta pathways containing significantly (p<0.05) many of the differentially expressed target genes were identified and listed in Table 3. In total, nine KEGG pathways were overrepresented and among these are “Wnt signaling pathway”, “Focal adhesion”, and “Calcium signaling pathway”. Moreover, three BioCarta pathways were identified as overrepresented among the differentially expressed target genes (Table 3) and these are “BTG family proteins and cell cycle regulation”, “Regulation of PGC-1a”, and, “NFAT and Hypertrophy of the heart”.

### Example 6

### Functional Annotation of the Differentially Expressed Target Genes

The DAVID bioinformatic resource (http://david.abcc.ncifcrf.gov/) was used to conduct a functional annotation analysis of the differentially expressed genes. Gene Ontology (GO) annotations including Biological Process (BP), Molecular Function (MF), and Cellular Component (CC) ¹⁷ were investigated, and significantly overrepresented (p<0.05) GO annotations were identified. The set of regulated genes was also investigated for significantly overrepresented (p<0.05) pathways among the genes using the KEGG (http://www.genome.jp/kegg) and BioCarta pathway (http://www.biocarta.com) databases.

### Example 7

### Introduction of miRNA-Encoding Nucleic Acid or Expression Constructs into Pluripotent Stem Cells

As discussed above, an aspect of the invention refers to a method which involves introducing into a stem cell or a progenitor cell (or a population of stem cells or progenitor cells) a miRNA-encoding nucleic acid. In some embodiments, a subject method involves introducing into a stem cell or a progenitor cell (or a population of stem cells or progenitor cells) one or more nucleic acids comprising nucleotide sequences (nucleotide constructs) encoding an miRNA. The following steps could be employed by someone skilled in the art to introduce an miRNA or miRNA expression construct into a hES cell. Present method is essentially an adaptation of the method suggested by Peerani, R. et al. (2007) and by Dharmacon for the introduction of small molecule RNAs into embryonic stem cells using their DharmaFECT™ transfection reagent. This approach could optionally be applied also to introduce miRNA analogues (see Example 9 below) into hES cells:
1. Culture desired hES cell line on irradiated mEF feeder layer in a medium such as Knockout DMEM (Invitrogen) supplemented with 20% KO Serum replacement (Invitrogen) and 4ng/ml basic FGF; passaging should be carried out every 3-4 days using manual dissection of hES colonies or enzymatic passaging using 0.1% Collagenase IV
2. 3-4 passages prior to use, hES cells should be switched to feeder-free conditions and passaged onto plates coated with 0.1% gelatine. The medium should be the same as in step 1, but it should be pre-conditioned by exposure to mEF cells for 24 hours prior to use.
3. Cells to be transfected should be passaged and replated at a density of ˜2×10³ per well in a 96 well tissue culture plate in a volume of ˜200 μl DharmaFECT 1 medium with 100 nM pre-synthesised miRNA
4. Cells can now be further passaged or assayed for activity of chosen miRNA

A further embodiment of this method involves monitoring and detection of miRNA and miRNA constructs in modified cell types, such as through the RNA extraction and 15 microarray method outlined in Example 2. Suitable methods also include detection by immunoassay or PCR-based methods the cardiac genetic markers described above (“Definitions”).

### Example 8

### Modulation of miRNA Activity Through the Use of miRNA Analogues

A further example includes the alteration of intracellular miRNA activity through the use of synthetic miRNA analogue oligonucleotides to promote a cardiac phenotype in undifferentiated or partially differentiated cell types. In this case, the method described in Example 7 could be employed to culture and transfect the stem cells with a suitable miRNA analogue.

### Example 9

### Detection of Cardiac Disease States Using miRNAs as Diagnostic Markers

A further example is the possible use of miRNA sequences as markers for cardiac disease, in particular the alteration of miRNA levels within diseased cells compared to a reference group. Tables 1.i) to 1.xiv) provides a list of up- and downregulated miRNAs. As apparent from the tables, the up- and downregulation of miRNAs depend highly on the developmental stage of the cells. Thus each developmental stage is characterized by a specific miRNA expression pattern, thereby assigning a miRNA fingerprint to a certain developmental stage. Thus, by using the miRNA expression patterns as listed in table 1i) to 1.xiv) as reference, a person skilled in the art is enabled to compare the expression pattern of one or more of the sequences provided in tables 1.i) to 1.xiv) the expression pattern in a sample and thereby detect irregularities or disease states in the sample. The methodology described in Example 2 could be adapted by someone skilled in the art to isolate and analyse miRNAs derived from tissue samples such as cardiac tissue.

### Example 10

### Isolation of Putative Cardiac or Cardiac-Like Cells Based on Alterations in miRNA Expression Levels

A further example of the invention is the use of miRNA to isolate cardiac or cardiac-like cells. Specifically, this relates to the analysis of expression levels of one or more miRNAs within a heterogeneous mixed cell population and the isolation of cells from the mixed population which display upregulated levels of the chosen miRNA(s), those cells being possible cardiac or cardiac-like cells. This could be achieved through the use of a reporter system such that emplyed by Kato, Y. et al. (2009) and Brown, B. D. et al. (2006) whereby indirect detection of an miRNA is achieved through the fusion of the UTR of a reporter gene to the target sequence of the miRNA. A suitable protocol for such an isolation method could be as follows and would be easily adaptable by someone skilled in the art:
1. Select miRNA of interest from list of those upregulated in CMC3w, CMC7w, FH or AH
2. Determine putative mRNA binding sites/sequences (e.g. as determined using a suitable database such as EMBL or www.microrna.org)
3. Prepare an expression construct using the method employed by Kato. Y. et al. (2009) as guidance; whereby a three-tandem repeat of a sequence with complete complementarity to the miRNA sequence under investigation is cloned into a suitable GFP reporter vector such that the translated target mRNA protein sequence will be GFP tagged.
4. Insert expression construct into actively dividing hES cell using method described in Example 7 or using a suitable electroporation or viral system as e.g. employed by Kato, Y. et al. (2009).
5. Culture hES cells as described in Example 7 post-transfection, optionally add one or more growth factors to induce cardiogenesis.
6. To detect GFP expression, wash cells x2 with PBS, then trypsinise for 3-5 minutes. Add sufficient cell culture medium to cells to quench trypsin and disperse into single cell suspension.
7. Analyse on suitable flow cytometer (FACS) to detect proportion of GFP-expressing cells and optionally sort into GFP⁺ and GFP⁻ fractions

In this case, cells expressing low levels of GFP will be those in which the subject miRNA is most highly expressed and in which it is actively repressing the expression of the target mRNA and thus the GFP reporter; high levels of GFP therefore conversely suggest low expression of the miRNA of interest. Thus it is possible to isolate cells expressing high levels of a chosen miRNA which may then be further analysed for markers of cardiogenesis.

Alternatively the expression level of the miRNA could be monitored directly by the following protocol:
1. Select miRNA of interest from list of those upregulated in CMC3w, CMC7w, FH or AH
2. Prepare an expression construct wherein the promoter region of the miRNA encoding sequence is inserted in-frame upstream of a fluorescent marker such as GFP
3. Insert expression construct into actively dividing hES cell using method described in Example 7 or using a suitable electroporation or viral system as e.g. employed by Kato, Y. et al. (2009).
5. Culture hES cells as described in Example 7 post-transfection, optionally add one or more growth factors to induce cardiogenesis.
6. To detect GFP expression, wash cells x2 with PBS, then trypsinise for 3-5 minutes. Add sufficient cell culture medium to cells to quench trypsin and disperse into single cell suspension.
7. Analyse on suitable flow cytometer (FACS) to detect proportion of GFP-expressing cells and optionally sort into GFP⁺ and GFP⁻ fractions

In this case, cells expressing high levels of GFP will most reasonably be those in which the subject miRNA is most highly expressed and in which the promoter driving the expression of the miRNA containing sequence will resemble the promoter driving the expression if the fluorescent marker. Thus, when the miRNA promoter is activated in cis the promoter driving the GFP and thereby yielding a fluorescent signal upon activation. Thus it is possible to isolate cells expressing certain (high or low) levels of a chosen miRNA which may then be further analysed for markers of cardiogenesis.

### DESCRIPTION OF THE DRAWINGS

FIG. 1. Experimental design and method for identification of miRNA target genes, showing the tissues from which the various miRNA samples were harvested and the general methodology used to identify miRNAs which are up or down regulated in each sample.

FIG. 2: A Venn diagram illustrating the number of overlapping up- and down-regulated miRNAs (A) and mRNAs (B) in the four cell- and tissue samples.

FIG. 3: Pie charts showing overrepresented GO-annotations for the target genes of differentially expressed miRNAs in the BP (panel A), MF (panel B) and CC (panel C) categories. Only differentially expressed target genes were included in the analysis.

FIG. 4: Table 1 i) miRNAs downregulated in CMC3w

FIG. 5: Table 1.ii) microRNAs upregulated in CMC3w

FIG. 6: Table 1.iii) microRNAs downregulated in CMCw7

FIG. 7: Table 1.iv) microRNAs upregulated in CMCw7

FIG. 8: Table 1.v) microRNAs downregulated in Foetal Heart (FH)

FIG. 9: Table 1.vi) microRNAs upregulated in Foetal Heart (FH)

FIG. 10: Table 1.vii) microRNAs downregulated in Adult Heart (AH)

FIG. 11: Table 1.viii) microRNAs upregulated in Adult Heart (AH)

FIG. 12: Table 1.ix) microRNAs upregulated in CMC3w & CMC7w

FIG. 13: Table 1.x) microRNAs downregulated in CMC3w and CMC7w

FIG. 14: Table 1.xi) microRNAs upregulated in CMC3w, CMC7w and FH and Table 1.xii) microRNAs downregulated in CMC3w, CMC7w and FH

FIG. 15: Table 1.xiii) microRNAs upregulated in CMC3w, CMC7w, FH and AH and Table 1.xiv) microRNAs down regulated in CMC3w, CMC7w, FH and AH

FIG. 16: List of novel miRNA sequences.

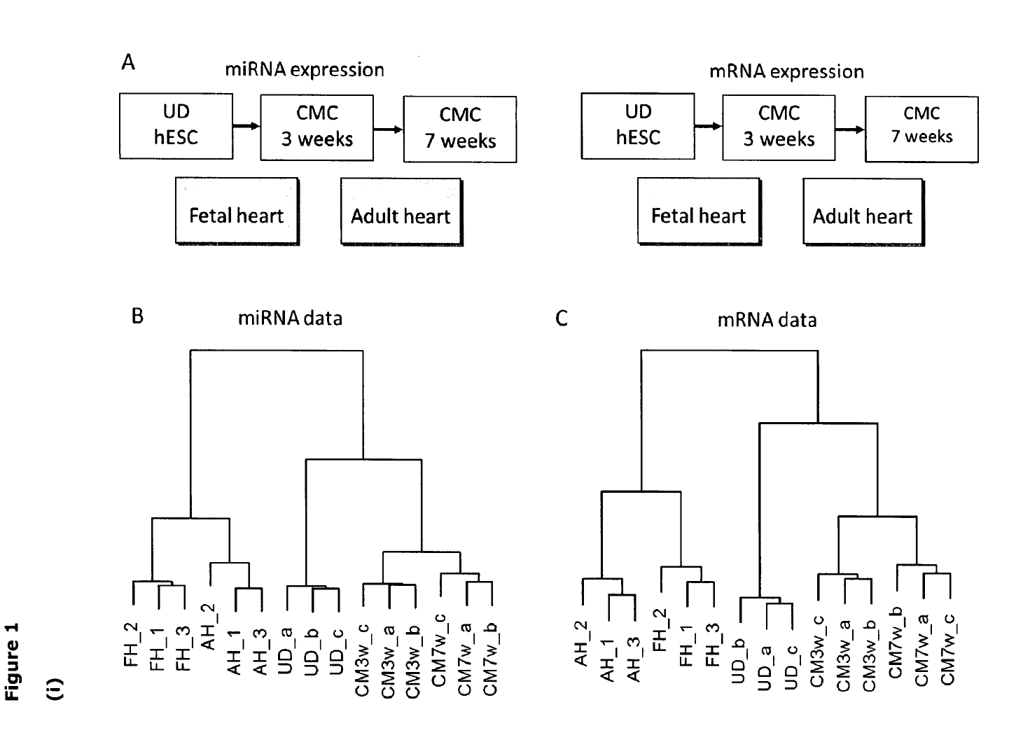

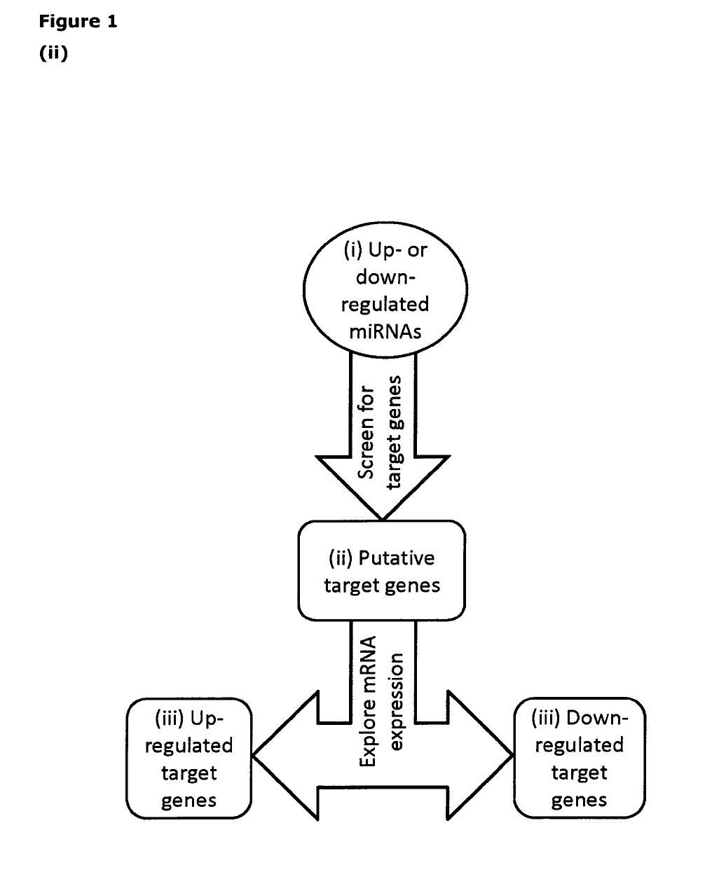

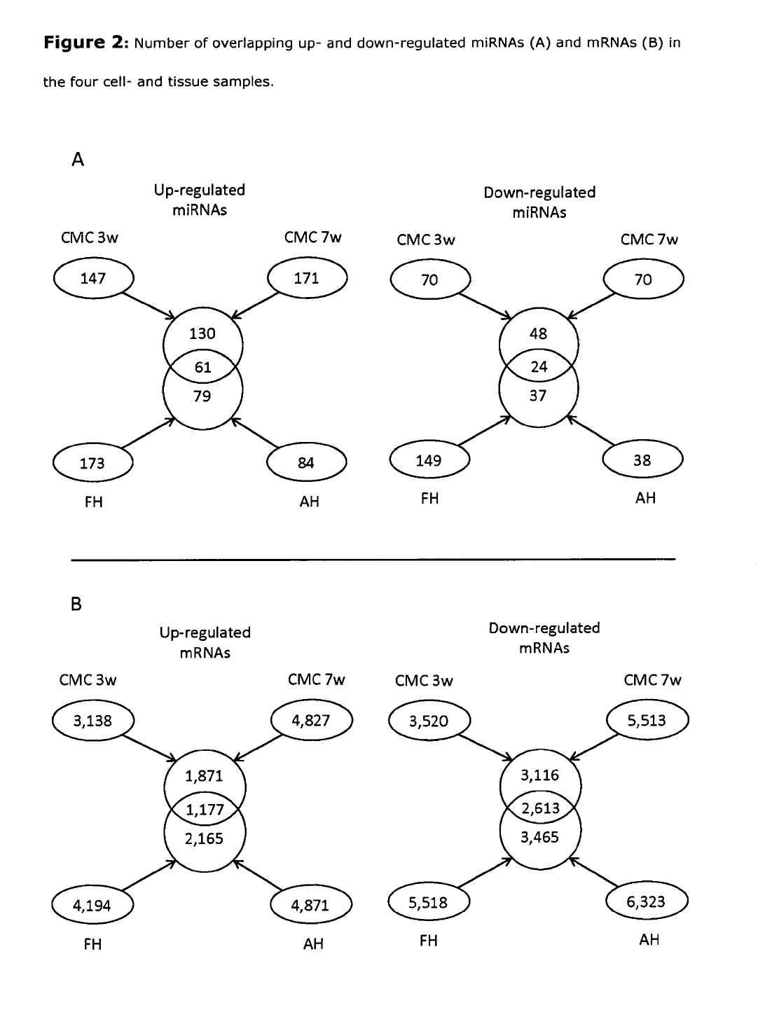

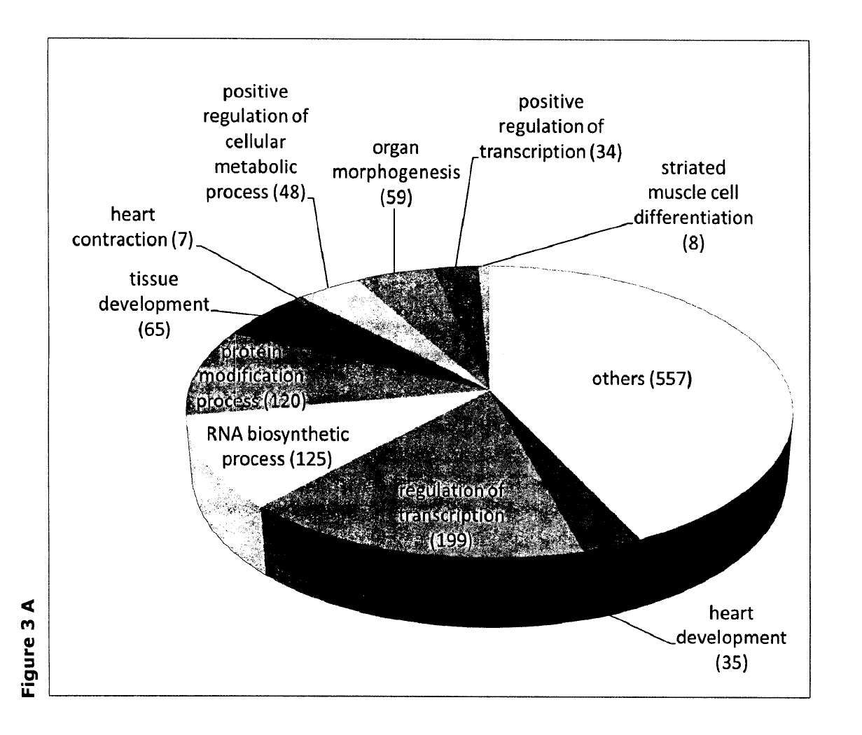

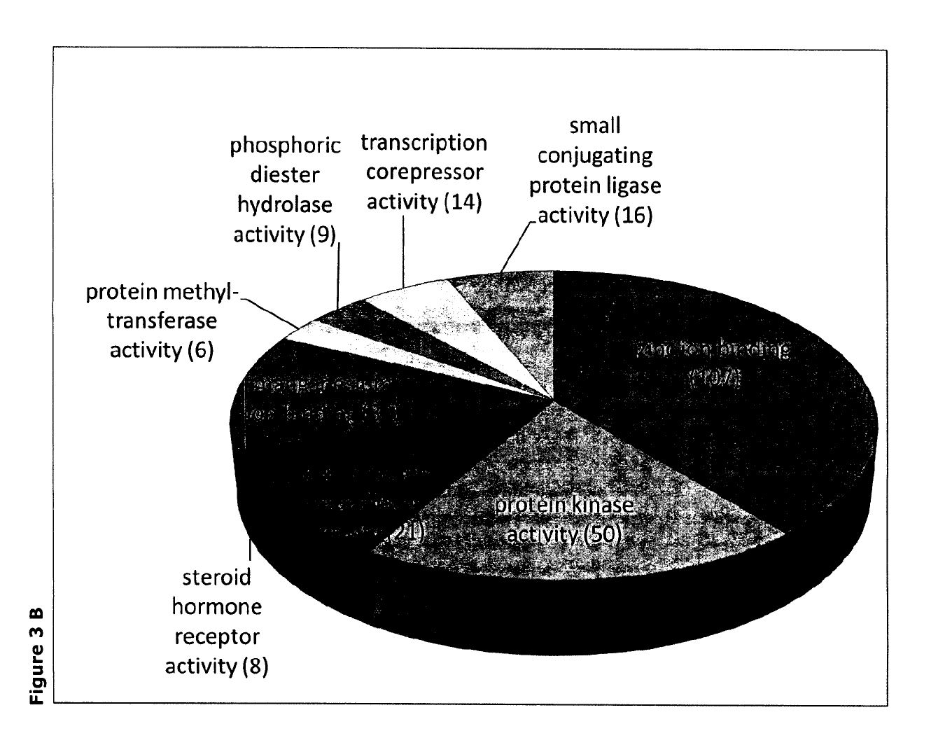

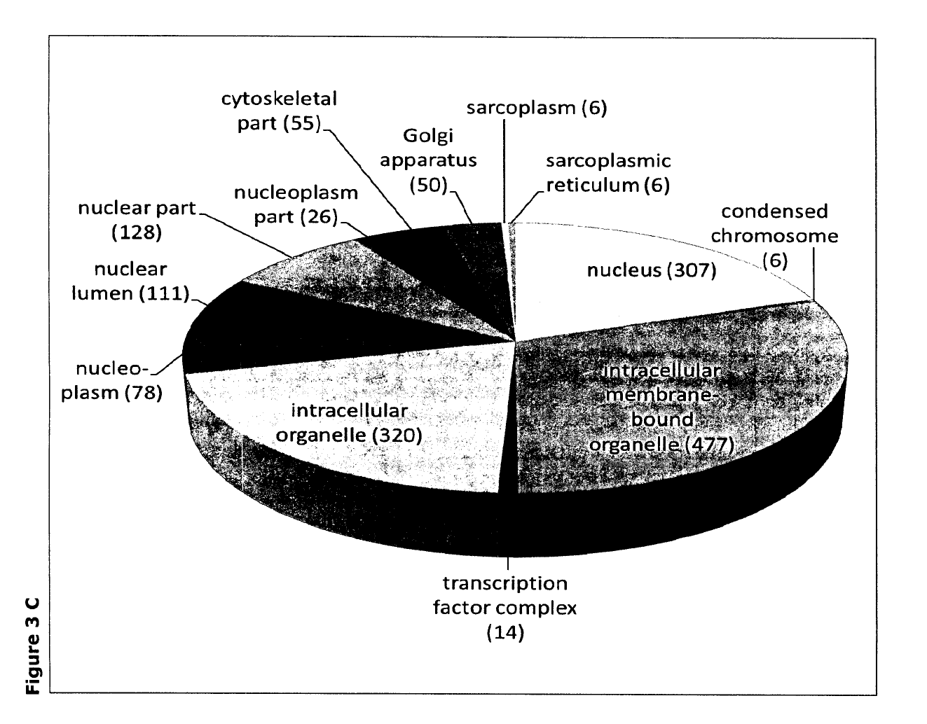

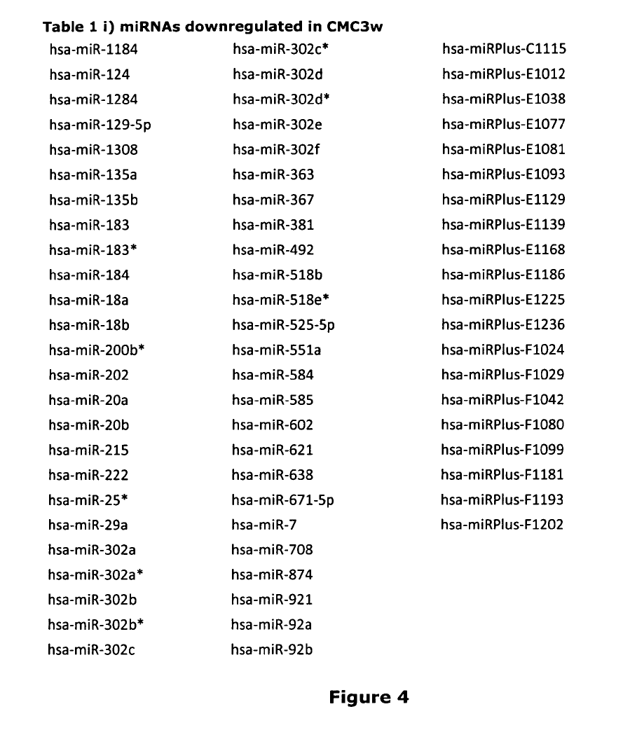

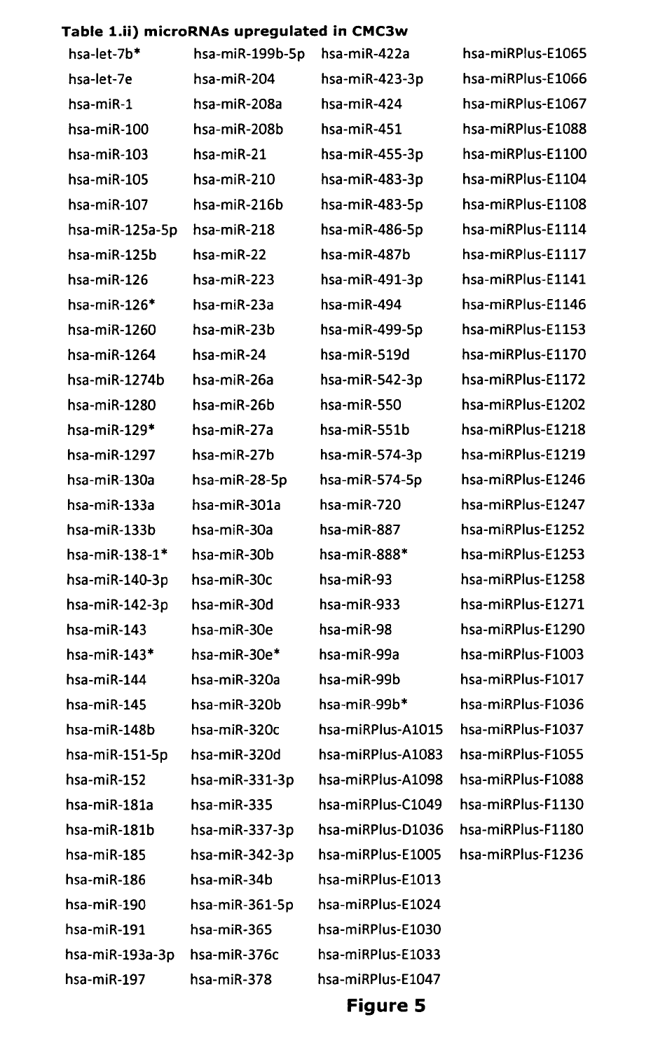

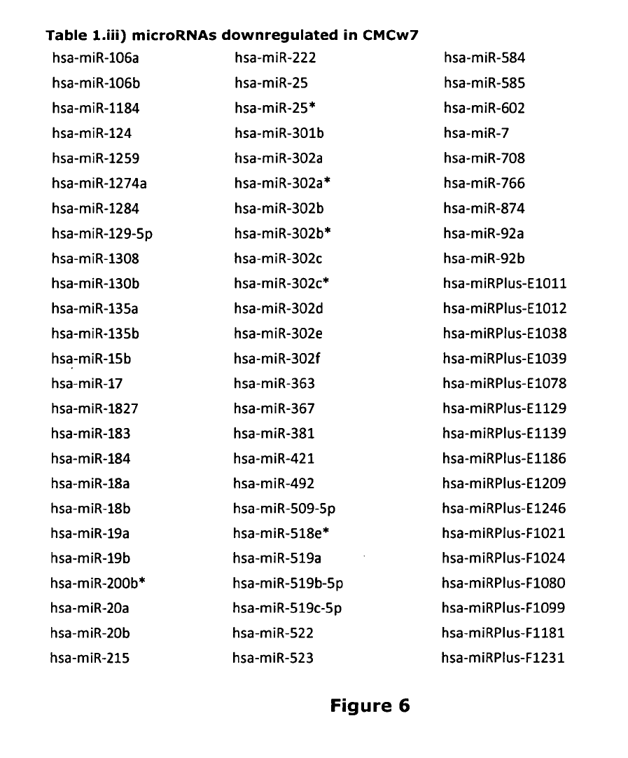

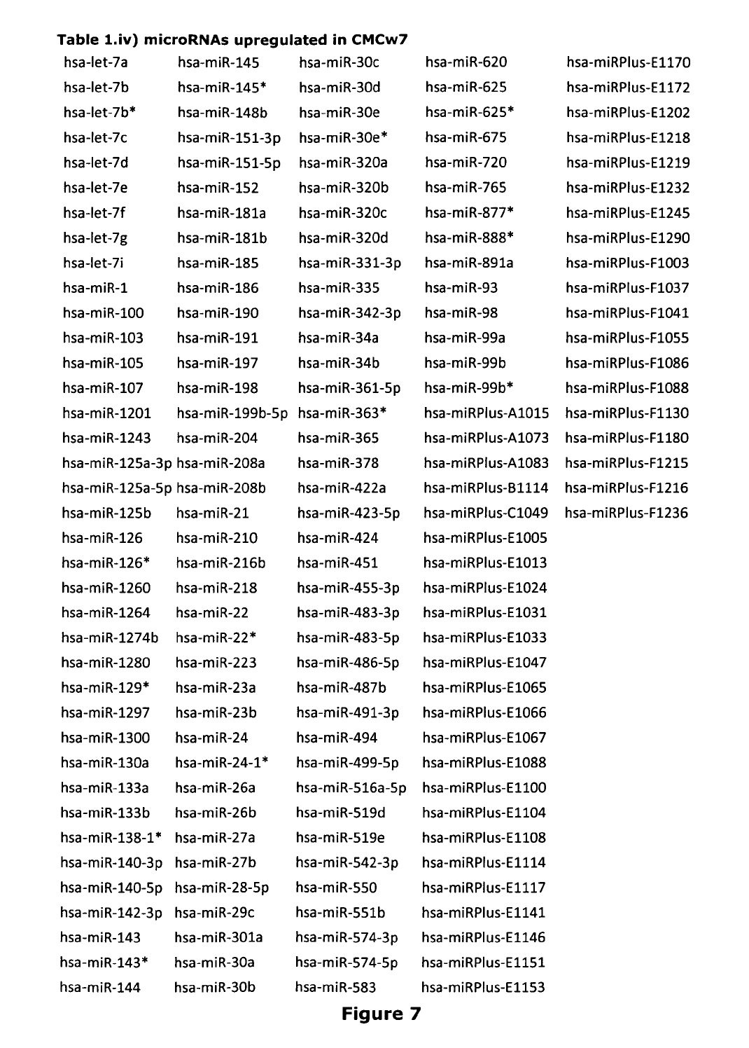

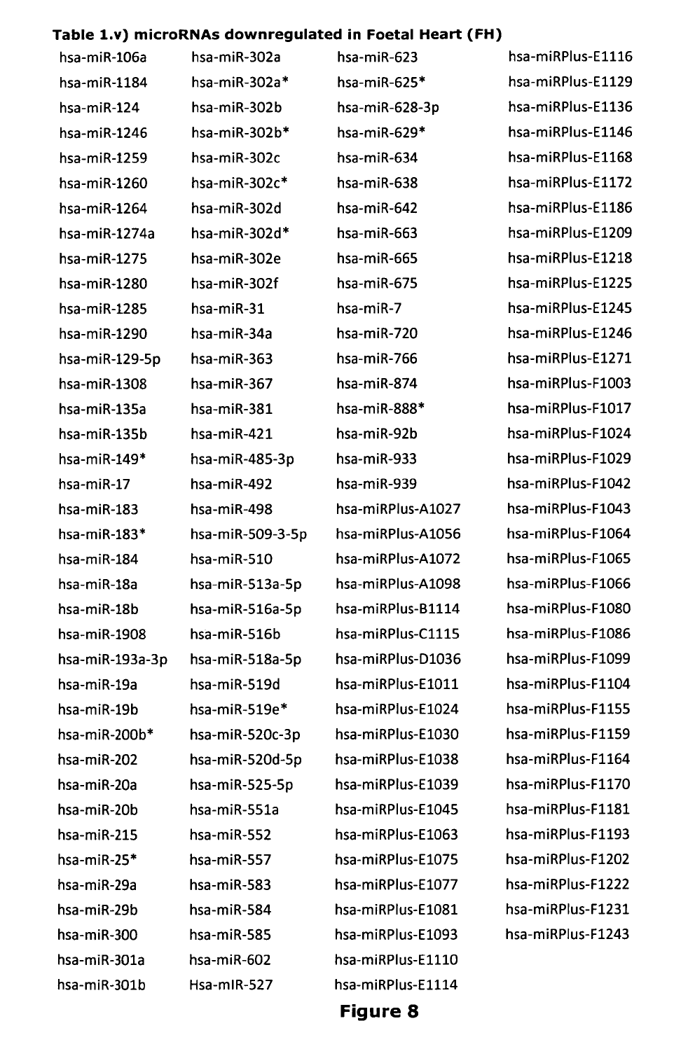

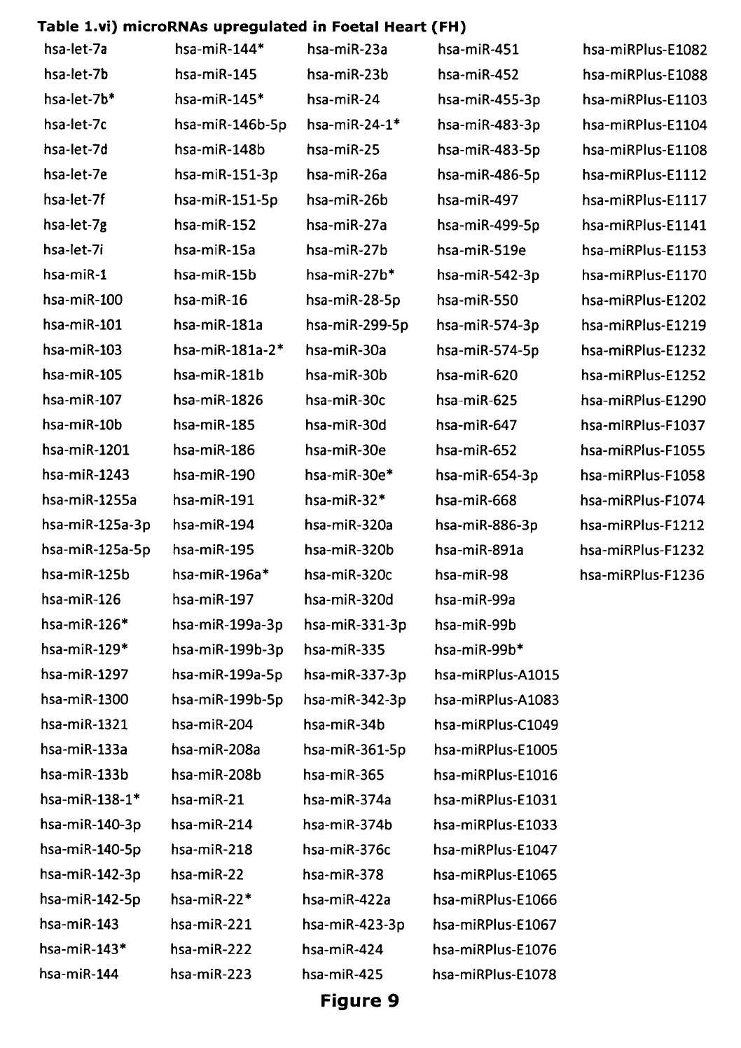

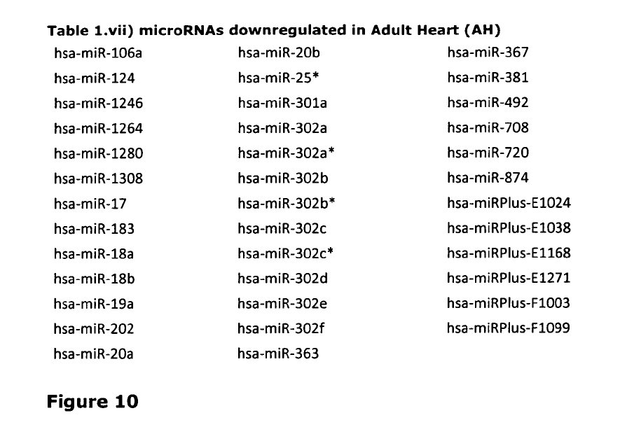

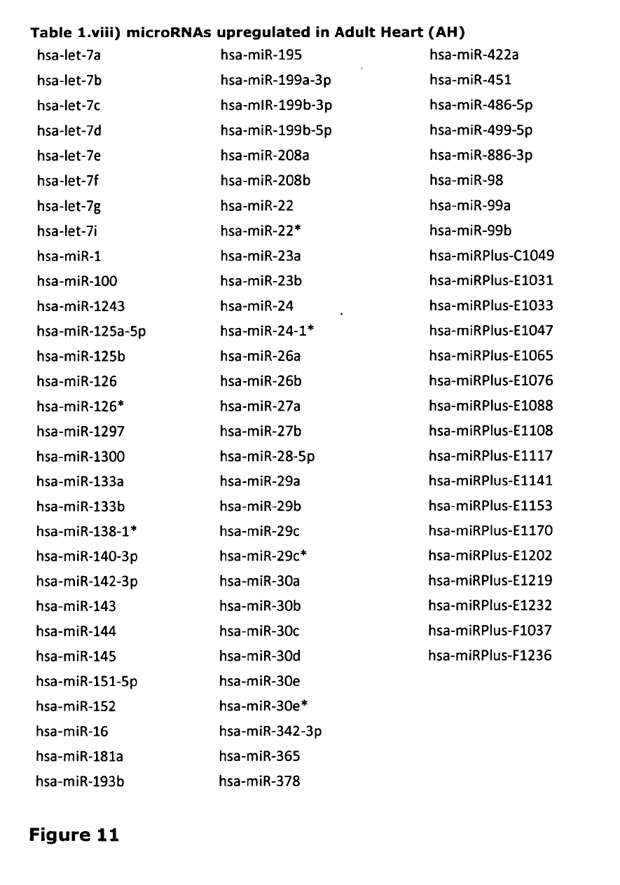

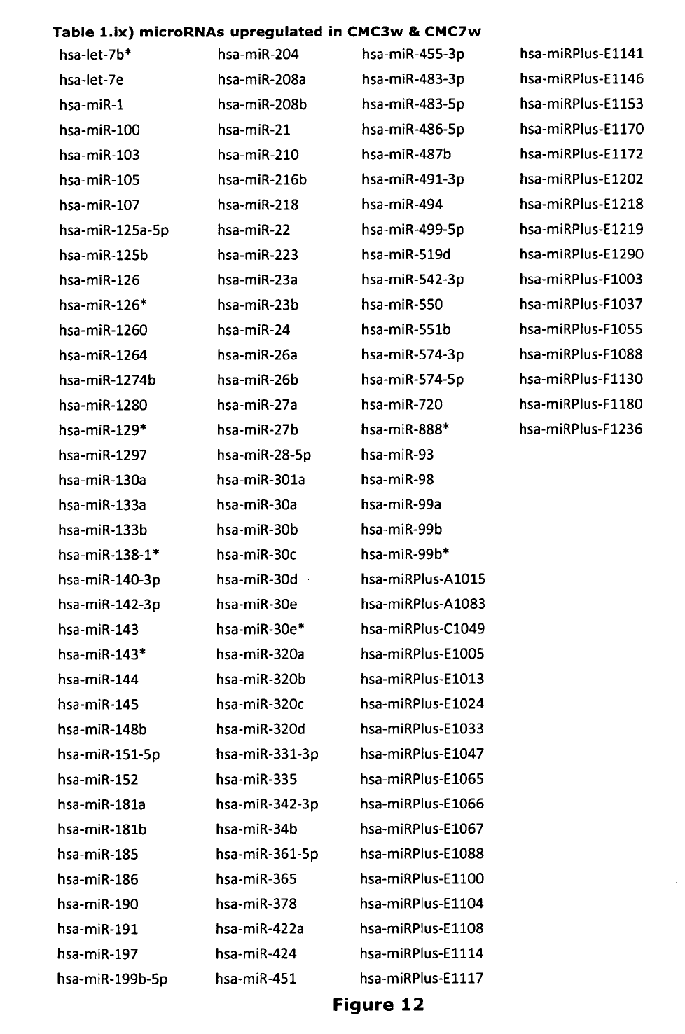

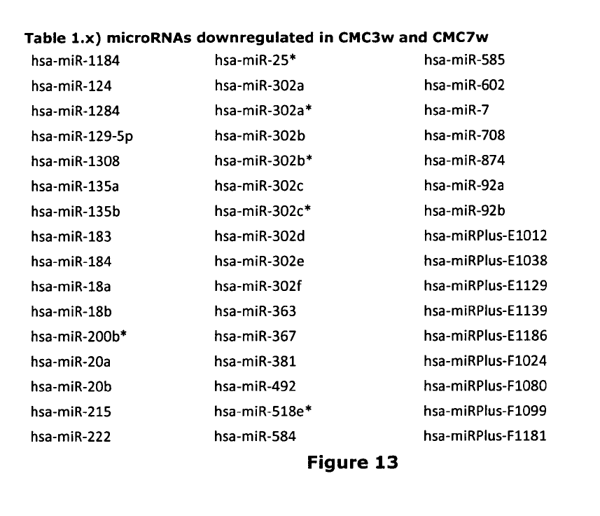

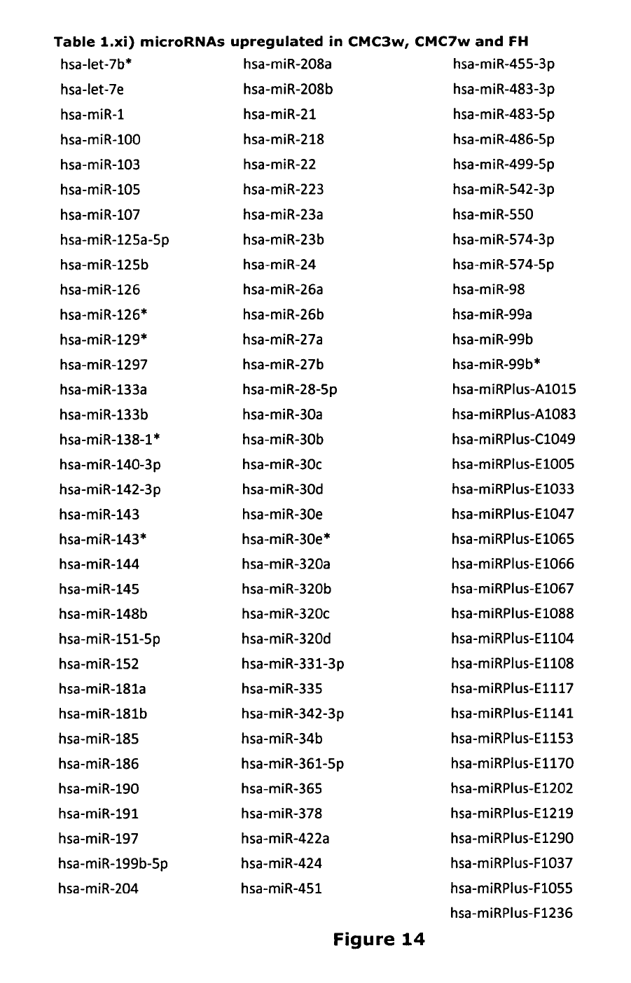

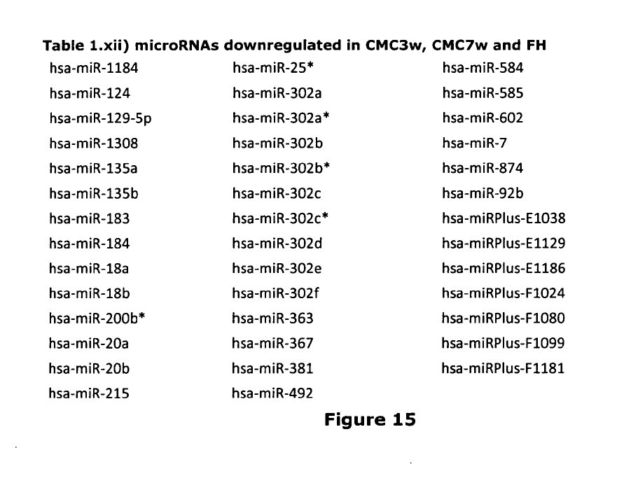

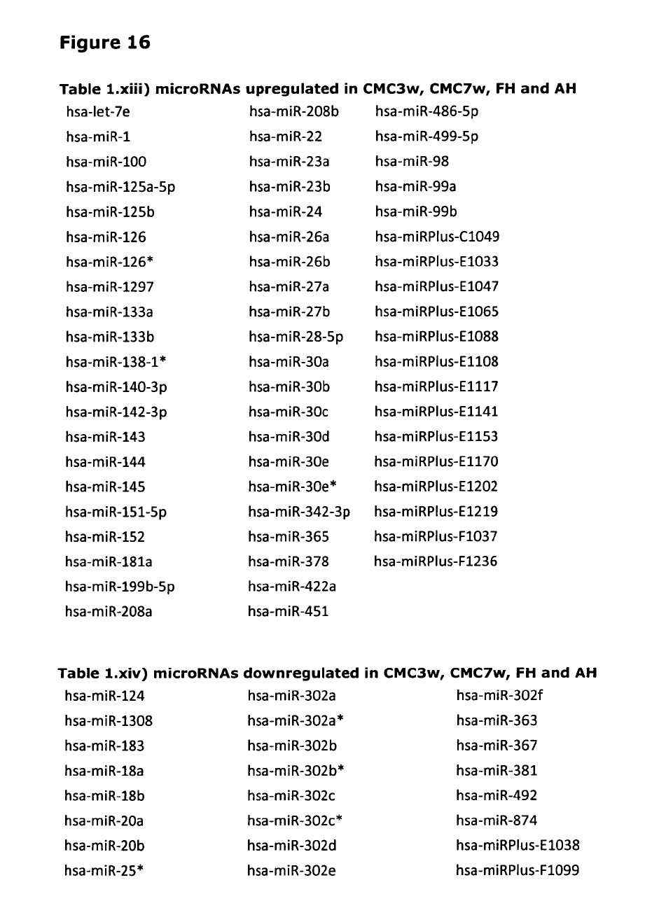

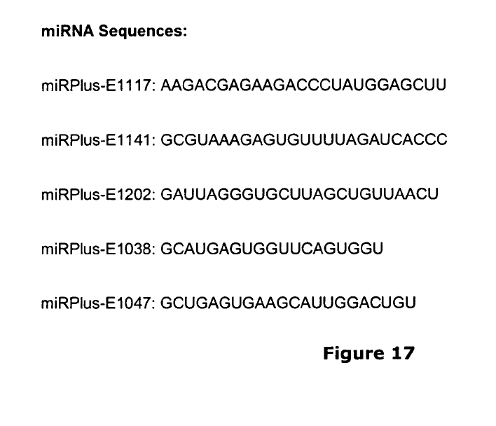

### REFERENCES

Bitter, G. A. et al. (1987) Expression and secretion vectors for yeast. Methods in Enzymology 153: 516-544

Brown, B. D. et al. (2006) Endogenous microRNA regulation suppresses transgene expression in hematopoetic lineages and enables stable gene transfer. Nat. Med. 12: 585-591

Flannery, J. G. et al. (1997) Efficient photoreceptor-targeted gene expression in vivo by recombinant adeno-associated virus. Proc. Natl. Acad. Sci. USA 94: 6916-6921

Freier, S. M. and Altmann, K. H. et al. (1997) The ups and downs of nucleic acid duplex stability: structure-stability studies on chemically modified DNA:RNA duplexes. Nucleic Acids Res. 25: 4429-4443

Hutvagner, G. and Zamore, P. D. et al. (2002) A microRNA in a multiple turnover RNAi enzyme complex. Science 297: 2056-2060

Kato, Y. et al. (2009) Real-time functional imaging for monitoring miR-133 during myogenic differentiation. Int. J. Biochem. Cell. Biol. 41: 2225-2231

Krützfeldt, J. et al. (2005) Silencing of microRNAs in vivo with “antagomirs”. Nature 438: 685-689

Lakshmipathy, U. et al. (2007) MicroRNA expression pattern of undifferentiated and differentiated human embryonic stem cells. Stem Cells Development 16: 1003-16

Miyoshi, H. et al. (1997) Stable and efficient gene transfer into the retina using an HIV-based lentiviral vector. Proc. Natl. Acad. Sci. USA 94: 10319-10323

Peerani, R. et al. (2007) Niche-mediated control of human embryonic stem cell self-renewal and differentiation. EMBO J. 26: 4744-4755

Robertson, B. et al. (2010) Specificity and functionality of microRNA inhibitors. Silence 1: 10

van Rooij, E. et al. (2006) A signature pattern of stress-responsive microRNAs that can evoke cardiac hypertrophy and heart failure. Proc. Natl. Acad. Sci. USA 103: 18255-18260

van Rooij, E. et al. (2007) Control of stress-dependent cardiac growth and gene expression by microRNA. Science 316: 575-579

Schwarz, D. S. et al. (2003) Asymmetry in the assembly of the RNAi enzyme complex. Cell 115: 199-208

Srivastava, D. et al. (1997) Regulation of cardiac mesodermal and neural crest development by the bHLH transcription factor dHAND. Nature Genetics 16: 154-160

Takahashi, K. et al. (2007) Induction of pluripotent stem cells from adult human fibroblasts by defined factors. Cell 131: 861-872

Thomson, J. A. et al. (1998) Embryonic stem cell lines derived from human blastocysts. (1998) Science 282: 1145-1147

Uhlmann, E. et al. (2001) Oligonucleotide technologies: synthesis, production, regulations and applications. Expert Opin. Biol. Ther. 1: 319-328

Yu, J. et al. (2007) Induced pluripotent stem cell lines derived from human somatic cells. Science 318: 1917-1920

Zhao, Y. et al. (2005) Serum response factor regulates a muscle-specific microRNA that targets Hand2 during cardiogenesis. Nature 436: 214-220

1. A method for promoting the development and/or maturation of stem cells towards cardiac progenitor or cardiomyocyte-like cells in vitro, by manipulating the intracellular level of one or more miRNAs.

2. The method according to claim 1, wherein at least one or more of the miRNAs belongs to the group consisting of the miRNAs which are upregulated and/or downregulated in CMC3w.

3. The method according to claim 1, wherein at least one or more of the miRNAs belongs to the group consisting of the miRNAs which are upregulated and/or downregulated in CMC7w.

4. The method according to claim 1, wherein at least one or more of the miRNAs belongs to the group consisting of the miRNAs which are upregulated and/or downregulated in foetal heart.

5. The method according to claim 1, wherein at least one or more of the miRNAs belongs to the group consisting of the miRNAs which are upregulated and/or downregulated in adult heart.

6. The method according to claim 1, wherein at least one or more of the miRNAs belongs to the group consisting of the miRNAs which are upregulated and/or downregulated in both CMC3w and CMC7w.

7. The method according to claim 1, wherein at least one or more of the miRNAs belongs to the group consisting of the miRNAs which are upregulated and/or downregulated in CMC3w, CMC7w and Foetal Heart (FH).

8. The method according to claim 1, wherein at least one or more of the miRNAs belongs to the group consisting of the miRNAs which are upregulated and/or downregulated in CMC3w, CMC7w, Foetal Heart (FH) and Adult Heart (AH).

9. The method according to claim 1 wherein the one or more miRNAs is selected from the group consisting of miR-378, miR-152, miR-1297, miR-208a, miR-208b, miR-451, miRPlus-E1117, miRPlus-E1141, miRPlus-E1202, miR-25*, miRPlus-E1038, and miRPlus-1047.

10. The method according to claim 1, wherein the development and/or maturation of stem cell towards cardiac or cardiomyocyte-like cells is done through up or downregulation of one or more endogenous miRNAs.

11. The method according to claim 1, wherein the development and/or maturation of stem cells towards cardiac or cardiomyocyte-like cells is done through the introduction into the cell of one or more miRNAs.

12. The method according to claim 1, wherein the development and/or maturation of stem cells towards cardiac or cardiomyocyte-like cells is done through introducing into a stem cell or progenitor cell a miRNA antagomir.

13. The method according to claim 1, wherein the miRNAs are introduced as an nucleotide construct such as an expression construct, as a solution of miRNAs and/or as viral constructs.

14. The method according to claim 1, wherein the miRNA is a nucleotide analogue.

15. The method according to claim 1, wherein the miRNA or miRNA analogue is an miRNA antagomir.

16. The method according to claim 1, wherein the nucleic acid encoding the miRNA is linked to an inducible promoter sequence.

17. The method according to claim 1, wherein where the miRNA material is a purified miRNA sequence.

18. A method for in vitro diagnosing cardiac abnormalities and disease states in a sample, comprising the detection of alterations in the levels of at least one miRNA within the diseased cells compared to a control group.

19. A method for detecting and characterising stem cells as cardiac or cardiac-like cells within a stem cell population, comprising:
a) analysing the cellular level of one or more miRNAs.

20. A method for isolating stem cells exhibiting cardiac or cardiac-like characteristics from a heterogeneous mixed-cell population using one or more microRNAs, comprising:
a) analysing the level of one or more intracellular miRNAs;
b) optionally, removing unwanted cells;
c) mechanically and/or enzymatically isolating the cells showing signs of cardiac differentiation; and
d) optionally, adding substances such as e.g. growth factors or differentiating agents to the growth medium to promote differentiation towards a cardiomyocyte cell fate.

21. The method according to claim 19 or claim 20, wherein at least one or more of the miRNAs belongs to the group consisting of the miRNAs which are upregulated and/or downregulated in CMC3w.

22. The method according to claim 19 or claim 20, wherein at least one, or more of the miRNAs belongs to the group consisting of the miRNAs which are upregulated and/or downregulated in CMC7w.

23. The method according to claim 19 or claim 20, wherein at least one or more of the miRNAs belongs to the group consisting of the miRNAs which are upregulated and/or downregulated in foetal heart.

24. The method according to claim 19 or claim 20, wherein at least one or more of the miRNAs belongs to the group consisting of the miRNAs which are upregulated and/or downregulated in adult heart.

25. The method according to claim 19 or claim 20, wherein at least one or more of the miRNAs belongs to the group consisting of the miRNAs which are upregulated and/or downregulated in CMC3w.

26. The method according to claim 19 or claim 20, wherein at least one or more of the miRNAs belongs to the group consisting of the miRNAs which are upregulated and/or downregulated in both CMC3w and CMC7w.

27. The method according to claim 19 or claim 20, wherein at least one or more of the miRNAs belongs to the group consisting of the miRNAs which are upregulated and/or downregulated in CMC3w, CMC7w and Foetal Heart (FH).

28. The method according to claim 19 or claim 20, wherein at least one or more of the miRNAs belongs to the group consisting of the miRNAs which are upregulated and/or downregulated in CMC3w, CMC7w, Foetal Heart (FH) and Adult Heart (AH).

29. The method according to claim 19, wherein the one or more miRNAs is selected from the group consisting of miR-378, miR-152, and miR-1297.

30. The method according to claim 1 or claim 19, wherein the stem cells are human pluripotent (hPS) stem cells.

31. The method according to claim 1 or claim 19, wherein the stem cells are induced pluripotent stem cells (iPS).

32. The method according to claim 1 or claim 19, wherein the stem cells are derived from a primary mammalian source.

33. The method according to claim 19, wherein the levels of miRNA markers are compared to a reference.

34. The method according to claim 19, wherein, the miRNA is an expression construct and wherein the miRNA encoding sequence, the promoter driving the expression of the miRNA sequence or the miRNA target sequence is fused to an exogenous reporter gene sequence.

35. A genetically modified cell, containing a nucleotide construct comprising a miRNA selected from any of the tables 1.i) to 1.xiv).

36. The genetically modified cell according to claim 35, wherein the miRNA encoding sequence or promoter or miRNA target sequence is fused to a reporter gene sequence.

37. The genetically modified cell according to claim 36, wherein the reporter gene sequence is of non-human origin.

38. The genetically modified cell according to claim 35, wherein the nucleotide construct is stably integrated into the cell genome.

39. The genetically-modified cell or progenitor cell according to claim 35, wherein the miRNA expression construct is transiently expressed.

40. An isolated cell obtained by the method as described in claim 1.

41. An isolated cell exhibiting cardiac or cardiac-like characteristics growing in an in vitro environment comprising miRNAs or miRNA antagomir.

42. The isolated cell according to claim 41 growing in an in vitro environment comprising one or more miRNAs selected from any of the tables 1.i) to 1.xiv) or miRNA antagomirs targeting miRNA selected from any of the tables 1.i) to 1.xiv).

43. The isolated cell according to claim 40, wherein the cell is a pluripotent stem cell, such as e.g., an induced pluripotent stem cell or a blastocyst derived stem cell.

44. The isolated cell according to claim 40, wherein the miRNA or miRNA antagomir is exogenously added or a synthetic construct.

45. An isolated in vitro derived stem cell according to claim 41, wherein the one or more miRNAs is exogenously added.

46. miRNA for use as a marker of cardiomyocyte differentiation.

47. One or more of the miRNA Sequences:

| | |
|---|---|
| miRPlus-E1117: | AAGACGAGAAGACCCUAUGGAGCUU, |
| miRPlus-E1141: | GCGUAAAGAGUGUUUUAGAUCACCC; |
| miRPlus-E1202: | GAUUAGGGUGCUUAGCUGUUAACU; |
| miRPlus-E1038: | GCAUGAGUGGUUCAGUGGU; |
| and | |
| miRPlus-E1047: | GCUGAGUGAAGCAUUGGACUGU. |

48. Use of any of the miRNA Sequences:
miRPlus-E1117: AAGACGAGAAGACCCUAUGGAGCUU;
miRPlus-E1141: GCGUAAAGAGUGUUUUAGAUCACCC;
miRPlus-E1202: GAUUAGGGUGCUUAGCUGUUAACU;
miRPlus-E1038: GCAUGAGUGGUUCAGUGGU;
miRPlus-E1047: GCUGAGUGAAGCAUUGGACUGU; and
for detecting, characterising, isolating, or differentiating hPS cells.

49. Use of any of the miRNA Sequences:
miRPlus-E1117: AAGACGAGAAGACCCUAUGGAGCUU;
miRPlus-E1141: GCGUAAAGAGUGUUUUAGAUCACCC;
miRPlus-E1202: GAUUAGGGUGCUUAGCUGUUAACU;
miRPlus-E1038: GCAUGAGUGGUUCAGUGGU;
miRPlus-E1047: GCUGAGUGAAGCAUUGGACUGU; and
in diagnostics, drug discovery, toxicity testing, or drug development.


SEQUENCE LISTING (5 sequences)

SEQ ID NO: 1 - RNA, 25 nt, Homo sapiens
aagacgagaagacccuauggagcuu

SEQ ID NO: 2 - RNA, 25 nt, Homo sapiens
gcguaaagaguguuuuagaucaccc

SEQ ID NO: 3 - RNA, 24 nt, Homo sapiens
gauuagggugcuuagcuguuaacu

SEQ ID NO: 4 - RNA, 19 nt, Homo sapiens
gcaugagugguucaguggu

SEQ ID NO: 5 - RNA, 22 nt, Homo sapiens
gcugagugaagcauuggacugu